(19) United States
(12) Patent Application Publication
(10) Pub. No.: US 2006/0281979 A1
(43) Pub. Date: Dec. 14, 2006

(54) Sensing device for sensing emergency situation having acceleration sensor and method thereof

(76) Inventors: Seung-Nam Kim, Anyang (KR); Hyuk-Jae Baik, Seoul (KR); Jae-Ryong Jung, Yongin (KR)

Correspondence Address: ST. ONGE STEWARD JOHNSTON & REENS, LLC, 986 BEDFORD STREET, STAMFORD, CT, 06905-5619, US

(21) Appl. No.: 11/251,305

(22) Filed: Oct. 14, 2005

(30) Foreign Application Priority Data
Jun. 9, 2005 (KR) 2005/49459

(51) Int. Cl. A61B 5/00; A61B 5/02; A61B 5/103

(52) U.S. Cl. 600/301; 600/595; 600/500; 600/485

(57) ABSTRACT

A sensing device for sensing an emergence situation having an acceleration sensor and a method thereof are provided. The sensing device includes acceleration sensing means, blood pressure/pulse sensing means, comparing means, communication means, and a controller. The accelerating sensing means senses acceleration generated due to movement of the body and the blood pressure/pulse sensing means senses a blood pressure and a pulse of a user. The comparing means compares acceleration, a blood pressure, and a pulse with a reference acceleration, a reference blood pressure, and a reference pulse, generates an acceleration abnormal signal, a blood pressure abnormal signal, and a pulse abnormal signal, and outputs an emergency situation signal. The communication means transmits the emergency situation signal to an external apparatus through a communication network and the controller controls the communication means to transmit the emergency situation signal to the external apparatus if an emergency situation signal is generated from the comparing means.

This application claims priority of pending Korean Patent Application No. 2005-0049459 filed on Jun. 9, 2005.

### Field of the Invention

The present invention relates to a sensing device for sensing an emergence situation having an acceleration sensor that senses an emergence situation from movements of the body of a user who carries the acceleration sensor with him and a method thereof.

### Description of the Related Art

An interactive diagnosis system is a system in which a doctor receives a signal transmitted from various sensors attached to a patient to make out a prescription and the state of a patient is sensed and transmitted to a remotely located doctor so that a doctor can properly diagnose and make out a prescription.

One of the interactive diagnosis systems, technologies for sensing emergence situations that might be generated to a patient have developed. There is Korean Patent Publication No. 1999-0064769 titled “Portable Automatic Alarm” for one of such technologies.

FIG. 1 is a circuit diagram of a portable automatic alarm of the related art.

A main machine 2 of the portable automatic alarm includes human body state sensors 4 for sensing a blood pressure and the pulse, a position checking device 6 for checking the position of a user, manipulation buttons 16, and a transmitter 10 for transmitting the position checked by the position checking device 6 to the outside.

A controller 8 analyzes a sensed signal inputted from the human body state sensors 4, and if it is judged that emergence situation happens as a result of analysis of the sensed signal, the controller 8 delivers a signal checked by the position checking device 6 to the transmitter 10 to transmit a position signal to the outside, thereby informing a user's emergency situation to the outside.

Since there are many variables besides a blood pressure and the pulse in a human body, it is considered that the blood pressure and the pulse do not necessarily coincide with an emergency situation in the case where an emergency situation is actually generated and thus it is difficult to understand an emergency situation accurately.

Also, since the human body state sensors of the related art sense an emergency situation of a human body periodically or intermittently, an emergency situation has not been sensed in real-time.

### SUMMARY OF THE INVENTION

An object of the present invention is to provide a sensing device for sensing an emergence situation having an acceleration sensor capable of sensing an emergency situation in a more direct manner by detecting acceleration generated due to movement of a human body while supplementing indirect measurement factors such as a blood pressure and the pulse in the case where an emergency situation is generated to a patient.

According to an aspect of the present invention, there is provided a sensing device for sensing an emergence situation including: acceleration sensing means worn on a body of a user, for sensing acceleration generated due to movement of the body; blood pressure/pulse sensing means for sensing a blood pressure and a pulse of a user; comparing means for comparing acceleration, a blood pressure, and a pulse inputted from the acceleration sensing means and the blood pressure/pulse sensing means with a reference acceleration, a reference blood pressure, and a reference pulse, generating an acceleration abnormal signal, a blood pressure abnormal signal, and a pulse abnormal signal if compared differences are greater than preset values, and outputting an emergency situation signal if both the acceleration abnormal signal and at least one of the blood pressure abnormal signal and the pulse abnormal signal exist; communication means for transmitting the emergency situation signal to an external apparatus through a communication network; and a controller for controlling the communication means to transmit the emergency situation signal to the external apparatus if the emergency situation signal is generated from the comparing means.

According to another aspect of the present invention, there is provided a sensing device for sensing an emergence situation having an acceleration sensor including: acceleration sensing means worn on a body of a user, for sensing acceleration generated due to movement of the body; comparing means for judging whether the acceleration generated from the acceleration sensing means exceeds a preset value; communication means for checking whether an emergency situation is true or not if the acceleration value is judged to exceed the preset value, and informing the emergency situation to an outside if the emergency situation is actually generated; and a controller for controlling the comparing means and the communication means.

The sensing device may further include a blood pressure/pulse measuring means connected with the comparing means, for measuring a blood pressure or a pulse of a user and inputting the measured blood pressure or the pulse to the controller, wherein when the acceleration exceeds the preset value and the blood pressure or the pulse exceeds the preset value, the emergency situation is determined true.

The sensing device may further include a storage means for storing the acceleration sensed by the acceleration sensing means.

The acceleration sensing means may sense acceleration with respect to movements of a user in x, y, and z-axes.

The sensing device may further include a global positioning system (GPS) receiver for receiving a position of a user from GPS satellites and the controller may control to transmit a position of a user received from the GPS receiver to an external apparatus.

According to yet another aspect of the present invention, there is provided a method for sensing an emergence situation having an acceleration sensor including the steps of: judging whether acceleration sensed by an acceleration sensor worn by a user exceeds a preset value; if the acceleration is judged to exceed the preset value, sensing whether a blood pressure or a pulse inputted to a blood pressure/pulse measuring means worn by a user exceeds a preset value; and if the blood pressure or the pulse is sensed to exceed the preset value, transmitting an emergency situation signal to an external apparatus through a communication network.

### BRIEF DESCRIPTION OF THE DRAWINGS

The above objects and other advantages of the present invention will become more apparent by describing in detail preferred embodiments thereof with reference to the attached drawings in which:

FIG. 1 is a circuit diagram of a portable automatic alarm of the related art;

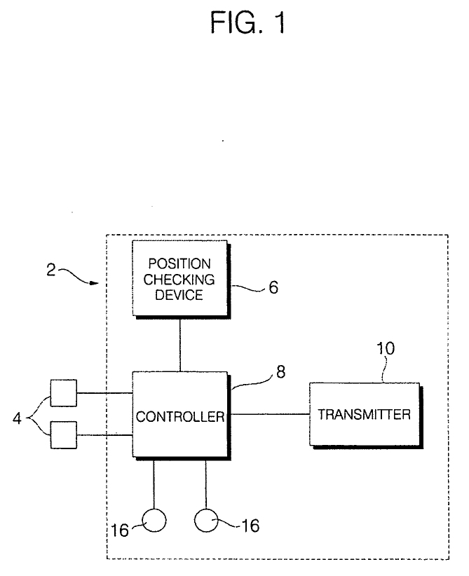

FIG. 2 is a block diagram of a sensing device according to an embodiment of the present invention;

FIG. 3 is a graph for acceleration measured by an acceleration sensor according to an embodiment of the present invention; and

FIG. 4 is a flowchart illustrating operations of a controller according to the present invention.

### DETAILED DESCRIPTION OF THE PREFERRED EMBODIMENT

Now, preferred embodiments of the present invention will be described in detail with reference to the accompanying drawings.

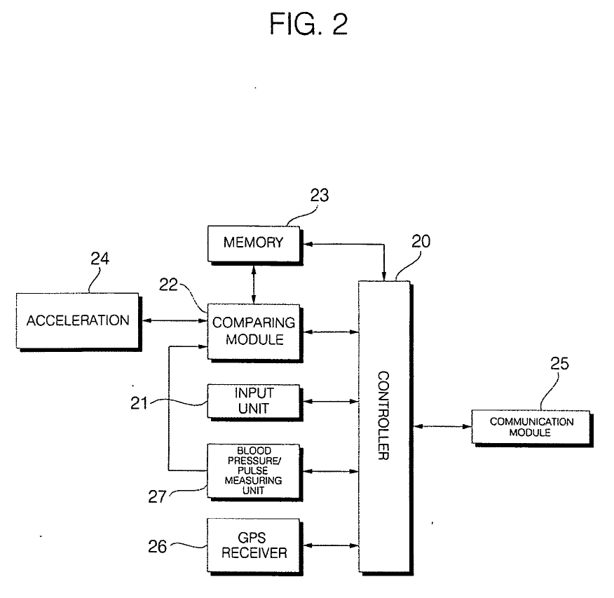

FIG. 2 is a block diagram of a sensing device according to an embodiment of the present invention.

A controller 20 may be a central processing unit (CPU) for controlling devices connected thereto. The controller 20 receives an input from an input unit 21 to change a set value and sets or resets a system. Also, the controller 20 is connected with a comparing module 22, which receives acceleration, a blood pressure, the pulse measured by an acceleration sensor 24 and a blood pressure/pulse measuring unit 27 to compare the received values with preset values, and generates an acceleration abnormal signal, a blood pressure abnormal signal, and a pulse abnormal signal if the received values exceed the preset values, and judges the present situation as a true emergency situation if both the acceleration abnormal signal and one of the blood pressure abnormal signal and the pulse abnormal signal are generated simultaneously, to transmit an abnormal state signal to the controller 20.

The acceleration, the blood pressure, and the pulse compared by the comparing module 22 are stored in a memory 23 by a program that operates the controller 20. At this point, in the case where the program is configured to store only acceleration, a blood pressure, and the pulse of more than a predetermined value, only the acceleration, the blood pressure, and the pulse of more than a predetermined value are stored in the memory 23. Acceleration data stored in this manner is read and used for understating the state of a patient and healing the patient.

Also, the controller 20 has a communication module 25 connected, for transmitting a signal to an external apparatus, e.g., a computer of a doctor. At this point, the communication module 25 is a device that can exchange data with the external apparatus through a wired/wireless network.

Also, the controller 20 has a GPS receiver 26 connected, for receiving the current position of a patient wearing the sensing device from GPS satellites to transmit the received current position of the patient to the controller 20, which then transmits the position of the patient to an external apparatus through the communication module 25 when an emergency situation of the patient occurs.

The acceleration sensor 24 is a device that can sense acceleration with respect to x, y, and z-axes, and values sensed by the acceleration sensor 24 are inputted to the comparing module 22. The comparing module 22 amplifies signals inputted from the acceleration sensor 24, converts the amplified signal into digital data to compare the data with a preset value, and transmits an abnormal signal to the controller 20 when the acceleration is greater than the preset value.

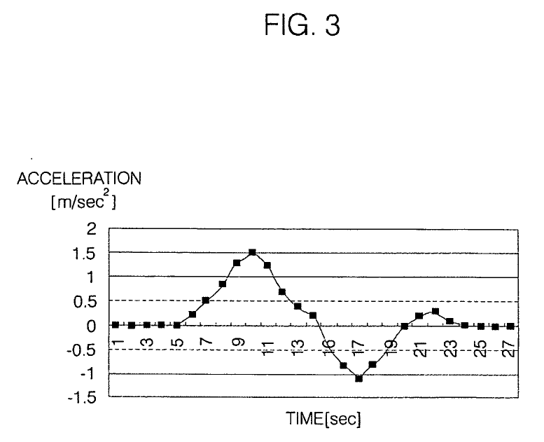

FIG. 3 is a graph for acceleration measured by an acceleration sensor according to an embodiment of the present invention.

In the case where an abrupt movement of a body is generated as when a patient falls down, the acceleration increases. On the contrary, in the case where a behavior of a patient is not constant, the acceleration fluctuates between negative values and positions values. The graph shows that a patient who wears the sensing device represents an abnormal symptom during an interval ranging from 5 to 24 seconds. If a value set to a threshold value of acceleration by the input unit 21 is 0.5 m/sec², the comparing module 22 transmits an abnormal signal to the controller 20 at the instant a user reaches the point of 7 seconds.

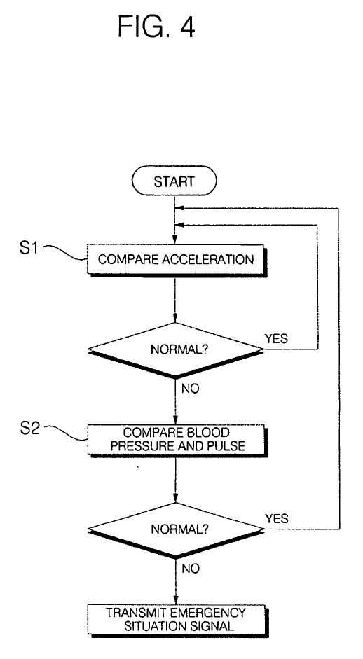

FIG. 4 is a flowchart illustrating operations of a controller according to the present invention.

The comparing module 22 judges whether acceleration is abnormal. If the acceleration is judged to be abnormal, the comparing module 22 inputs an acceleration abnormal signal to the controller 20 (S1). If the acceleration abnormal signal is inputted, the controller 20 inputs a blood pressure and the pulse measured by the blood pressure/pulse measuring unit 27 to the comparing module 22. If the inputted blood pressure and pulse exceed the preset values (S2), the comparing module 22 outputs an emergency situation signal to the controller 20. On the contrary, if the inputted blood pressure and pulse do not exceed the preset values, the comparing module 22 judges that a patient simply makes an abrupt motion and so it is a normal state.

If an emergency signal is inputted from the comparing module 22, the controller 20 controls the communication module 25 to transmit an emergency situation signal to an external apparatus, i.e., a computer of a doctor. At this point, the controller 20 reads the current position of a patient from the GPS receiver 26 and controls the communication module 25 to transmit the current position together with the emergency situation signal.

According to the present invention, an abrupt motion of a patient wearing the acceleration sensor is sensed and an abnormal state of the patient is informed to the outside, so that the abnormal state of the patient can be informed to the outside in a more direct and swift manner.

While the present invention has been described in detail, it should be understood that various changes, substitutions and alterations can be made hereto without departing from the spirit and scope of the invention as defined by the appended claims.

What is claimed is:

1. A sensing device for sensing an emergence situation comprising:
acceleration sensing means worn on a body of a user, for sensing acceleration generated due to movement of the body;
blood pressure/pulse sensing means for sensing a blood pressure and a pulse of a user;
comparing means for comparing acceleration, a blood pressure, and a pulse inputted from the acceleration sensing means and the blood pressure/pulse sensing means with a reference acceleration, a reference blood pressure, and a reference pulse, generating an acceleration abnormal signal, a blood pressure abnormal signal, and a pulse abnormal signal if compared differences are greater than preset values, and outputting an emergency situation signal if both the acceleration abnormal signal and at least one of the blood pressure abnormal signal and the pulse abnormal signal exist;
communication means for transmitting the emergency situation signal to an external apparatus through a communication network; and
a controller for controlling the communication means to transmit the emergency situation signal to the external apparatus if the emergency situation signal is generated from the comparing means.

2. The sensing device of claim 1, further comprising a storage means for storing the acceleration sensed by the acceleration sensing means.

3. The sensing device of claim 1, wherein the acceleration sensing means senses acceleration with respect to movements of a user in x, y, and z-axes.

4. The sensing device of claim 1, further comprising: a GPS (global positioning system) receiver for receiving a position of a user from GPS satellites, the controller controlling to transmit a position of a user received from the GPS receiver to an external apparatus.

5. A sensing device for sensing an emergence situation having an acceleration sensor comprising:
acceleration sensing means worn on a body of a user, for sensing acceleration generated due to movement of the body;
comparing means for judging whether the acceleration generated from the acceleration sensing means exceeds a preset value;
communication means for checking whether an emergency situation is true or not if the acceleration value is judged to exceed the preset value, and informing the emergency situation to an outside if the emergency situation is actually generated; and
a controller for controlling the comparing means and the communication means.

6. The sensing device of claim 5, further comprising a storage means for storing the acceleration sensed by the acceleration sensing means.

7. The sensing device of claim 5, wherein the acceleration sensing means senses acceleration with respect to movements of a user in x, y, and z-axes.

8. The sensing device of claim 5, further comprising: a GPS (global positioning system) receiver for receiving a position of a user from GPS satellites, the controller controlling to transmit a position of a user received from the GPS receiver to an external apparatus.

9. The sensing device of claim 5, further comprising a blood pressure/pulse measuring means connected with the comparing means, for measuring a blood pressure or a pulse of a user and inputting the measured blood pressure or pulse to the controller, wherein when the acceleration exceeds the preset value and the blood pressure or the pulse exceeds the preset value, the emergency situation is determined true.

10. A method for sensing an emergence situation having an acceleration sensor comprising the steps of:
judging whether acceleration sensed by an acceleration sensor worn by a user exceeds a preset value;
if the acceleration is judged to exceed the preset value, sensing whether a blood pressure or a pulse inputted to a blood pressure/pulse measuring means worn by a user exceeds a preset value; and
if the blood pressure or the pulse is sensed to exceed the preset value, transmitting an emergency situation signal to an external apparatus through a communication network.
